(19) United States
(12) Patent Application Publication
(10) Pub. No.: US 2026/0224272 A1
(43) Pub. Date: Aug. 6, 2026

(54) PROBE DETECTION FOR A MULTICHANNEL rf ABLATION SYSTEM

(71) Applicant: Boston Scientific Scimed, Inc, Maple Grove, MN (US)

(72) Inventors: Raul Serrano Carmona, Los Angeles, CA (US); Alexander Pruitt, San Jose, CA (US); Jin Yu, Valley Village, CA (US); James Sievert, Bradenton, FL (US); Michael J Lyden, Shoreview, MN (US)

(21) Appl. No.: 19/531,286

(22) Filed: Feb. 5, 2026

Related U.S. Application Data
(60) Provisional application No. 63/754,367, filed on Feb. 5, 2025.

(51) Int. Cl. A61B 18/12; A61B 18/00

(52) U.S. Cl. CPC A61B 18/1206; A61B 2018/00178; A61B 2018/00577; A61B 2018/00702; A61B 2018/00875; A61B 2018/1253; A61B 2018/126

(57) ABSTRACT

Systems and methods for probe detection in multichannel radiofrequency (RF) tissue ablation systems are described. The systems include a RF generator configured to be coupled to one or more RF probes forming one or more separate channels and having one or more electrodes each connectable to the RF generator via a separate electrical pin. The systems are configured to measure impedance between different electrical pins of the RF generator by opening and closing relays, and based on the measured impedance, determine a configuration of the RF probe connected to the RF generator by comparing the measured impedance to allowed ranges, and determine to which electrical pin(s) the RF probe is connected.

### CROSS REFERENCE TO RELATED APPLICATIONS

This application claims priority to U.S. Provisional Patent Application No. 63/754,367 entitled, “PROBE DETECTION FOR A MULTICHANNEL RF ABLATION SYSTEM,” filed Feb. 5, 2025, which is hereby incorporated by reference in its entirety.

### TECHNICAL FIELD

The present invention relates to tissue ablation systems and methods of operating the tissue ablation systems. More specifically, the present invention relates to multichannel radiofrequency tissue ablation systems and methods of probe detection in the multichannel radiofrequency tissue ablation systems.

### BACKGROUND

Radiofrequency energy has been used to ablate tissue at various locations within the body. For example, radiofrequency probes have been used to ablate tissue within the heart to treat atrial fibrillation. Radiofrequency probes have also been used to ablate nerves to facilitate treatment of facet joint pain or joint pain within the knees, shoulders, elbows, hips, or ankles.

A multichannel radiofrequency tissue ablation system includes multiple channels and can perform simultaneous thermal ablations using more than one monopolar or bipolar radiofrequency probe. Traditional multichannel radiofrequency tissue ablation systems rely on manual configuration and verification of probe types and connections. This approach introduces potential for error, requires additional procedural steps, and lacks automated safeguards against incorrect setup. Without automated detection capabilities, the system cannot prevent improper probe configurations that can lead to incorrect therapy delivery.

Therefore, there is a need in the art to develop systems and methods that enable automatic detection and verification of probe configurations in multichannel radiofrequency tissue ablation systems. Such systems would need to identify probe types, determine wiring configurations, and verify proper connections without user intervention, thereby ensuring safe and correct therapy delivery.

### SUMMARY

The present invention addresses the need for automated probe detection in multichannel radiofrequency tissue ablation systems through impedance measurement and analysis techniques. The tissue ablation system employs impedance testing and measuring between electrical contacts or pins in connection ports to automatically determine probe configurations without requiring manual determination and verification steps.

In one aspect, the system provides probe detection through coordinated operation of switching elements and impedance measurements. The system includes a radiofrequency generator comprising a power source and a controller operatively coupled to the power source and having a processor and a memory element. The generator includes a series of electrical pins that connect to various probe configurations. The processor executes program instructions that cause the controller to perform systematic impedance measurements between different electrical pins using controlled switching sequences.

The tissue ablation system determines probe configurations and connections by comparing measured impedance values against known reference values. For example, for monopolar probes, the system automatically detects the presence of a single active electrode connection. For bipolar probes, the system automatically detects and identifies the paired electrode connections and determines whether they are configured for single-channel or dual-channel operation. The system can also detect the presence of test equipment through characteristic impedance values.

Through sequencing of impedance measurements and switching operations, the system can build a complete map of probe connections and configurations. This enables automatic system configuration for appropriate therapy delivery while preventing improper setups that can compromise treatment safety or efficacy. The detection process operates continuously during system operation, providing real-time verification of probe configurations throughout the treatment process.

In Example 1, a tissue ablation system is provided. The tissue ablation system comprises a radiofrequency generator having a power source and a controller operatively coupled to the power source and having a processor and a memory element. The radiofrequency generator has a plurality of connector ports, wherein each of the plurality of connector ports has a plurality of electrical pins. The system further comprises a first radiofrequency probe electrically coupled to a first one of the plurality of connector ports of the radiofrequency generator to form a first channel and having first and second electrodes associated therewith, wherein each of the first and second electrodes are connected to the radiofrequency generator via a corresponding one of the plurality of electrical pins. The processor is also configured to execute program instructions stored in the memory element, such that the program instructions, when executed, cause the processor to: measure an impedance between different electrical pins of one or more of the plurality of connector ports, and based on the measured impedance, determine a configuration of the first radiofrequency probe electrically coupled to the radiofrequency generator.

In Example 2, the tissue ablation system of Example 1, wherein, to determine the configuration of the first radiofrequency probe, the processor is configured to determine whether the first radiofrequency probe is one of the following probe configurations: a monopolar radiofrequency probe utilizing the first electrode and a common return electrode; a bipolar radiofrequency probe coupled to the radiofrequency generator to form one channel and utilizing the first and second electrodes as a bipolar pair of electrodes; a bipolar radiofrequency probe utilizing two monopolar radiofrequency electrodes, wherein one of the two monopolar radiofrequency electrodes is connected to the first electrode and the other of the two monopolar electrodes is connected to the second electrode to form a bipolar pair where current flows between the first and second electrodes; or a test probe.

In Example 3, the tissue ablation system of Example 2, wherein the test probe has a predetermined impedance value.

In Example 4, the tissue ablation system of any of Examples 2-3, wherein the processor is further configured to compare the measured impedance to one or more reference impedance values to determine the probe configuration of the first radiofrequency probe.

In Example 5, the tissue ablation system of any of Examples 1-4, wherein the processor is further configured to determine, based on the measured impedance, to which one of the plurality of electrical pins the one or more of the first and second electrodes are connected.

In Example 6, the tissue ablation system of any of Examples 1-5, further comprising: a second radiofrequency probe electrically coupled to a second one of the plurality of connector ports of the radiofrequency generator to form a second channel and having third and fourth electrodes associated therewith, wherein each of the third and fourth electrodes are connected to the radiofrequency generator via a corresponding one of the plurality of electrical pins.

In Example 7, the tissue ablation system of Example 6, wherein to determine the configuration of the second radiofrequency probe, the processor is configured to determine whether the second radiofrequency probe is one of the following probe configurations: a monopolar radiofrequency probe utilizing the third electrode; a bipolar radiofrequency probe coupled to the radiofrequency generator to form one channel and utilizing the third and fourth electrodes as a bipolar pair of electrodes; or a test probe.

In Example 8, the tissue ablation system of any of Examples 6 or 7, wherein the processor is further configured to: compare the measured impedance to one or more reference impedance values to determine the probe configuration of the second radiofrequency probe.

In Example 9, the tissue ablation system of any of Examples 1-8, wherein the processor is further configured to: determine, based on the measured impedance, to which one of the plurality of electrical pins the one or more of the third and fourth electrodes are connected.

In Example 10, the tissue ablation system of any of Examples 1-9, wherein the radiofrequency generator comprises switching elements.

In Example 11, the tissue ablation system of Example 10, wherein the processor is configured to execute program instructions to open and close the switching elements when measuring impedance between different electrical pins.

In Example 12, the tissue ablation system of Example 1, wherein the power source is configured to deliver radiofrequency power to one of the first and second electrodes.

In Example 13, the tissue ablation system of Example 12, wherein the power is delivered via the first one of the plurality of connector ports and the corresponding one of the plurality of electrical pins.

In Example 14, the tissue ablation system of any of Examples 1 or 12-13, wherein the radiofrequency power is sufficient to provide controlled heating of tissue surrounding the at least one electrode during a surgical procedure.

In Example 15, the tissue ablation system of any of Examples 10 or 11, wherein the switching element is configured to control radiofrequency power delivery.

In Example 16, a tissue ablation system is provided. The tissue ablation system comprises a radiofrequency generator having a power source and a controller operatively coupled to the power source and having a processor and a memory element. The radiofrequency generator has a plurality of connector ports, wherein each of the plurality of connector ports has a plurality of electrical pins. The system further comprises a first radiofrequency probe electrically coupled to a first one of the plurality of connector ports of the radiofrequency generator to form a first channel and having first and second electrodes associated therewith, wherein each of the first and second electrodes are connected to the radiofrequency generator via a corresponding one of the plurality of electrical pins. The power source can be configured to deliver radiofrequency power to one of the first and second electrodes via the first one of the plurality of connector ports and the corresponding one of the plurality of electrical pins, the radiofrequency power being sufficient to provide controlled heating of tissue surrounding the at least one electrode during a surgical procedure. The processor is also configured to execute program instructions stored in the memory element, such that the program instructions, when executed, cause the processor to: measure an impedance between different electrical pins of one or more of the plurality of connector ports, and based on the measured impedance, determine a configuration of the first radiofrequency probe electrically coupled to the radiofrequency generator.

In Example 17, the tissue ablation system of Example 16, wherein, to determine the configuration of the first radiofrequency probe, the processor is configured to determine whether the first radiofrequency probe is one of the following probe configurations: a monopolar radiofrequency probe utilizing the first electrode and a common return electrode; a bipolar radiofrequency probe coupled to the radiofrequency generator to form one channel and utilizing the first and second electrodes as a bipolar pair of electrodes; a bipolar radiofrequency probe utilizing two monopolar radiofrequency electrodes, wherein one of the two monopolar radiofrequency electrodes is connected to the first electrode and the other of the two monopolar electrodes is connected to the second electrode to form a bipolar pair where current flows between the first and second electrodes; or a test probe.

In Example 18, the tissue ablation system of Example 17, wherein the test probe has a predetermined impedance value.

In Example 19, the tissue ablation system of Example 17, wherein the processor is further configured to compare the measured impedance to one or more reference impedance values to determine the probe configuration of the first radiofrequency probe.

In Example 20, the tissue ablation system of Example 16, wherein the processor is further configured to determine, based on the measured impedance, to which one of the plurality of electrical pins the one or more of the first and second electrodes are connected.

In Example 21, the tissue ablation system of Example 16, further comprising:
a second radiofrequency probe electrically coupled to a second one of the plurality of connector ports of the radiofrequency generator to form a second channel and having third and fourth electrodes associated therewith, wherein each of the third and fourth electrodes are connected to the radiofrequency generator via a corresponding one of the plurality of electrical pins.

In Example 22, the tissue ablation system of Example 21, wherein to determine the configuration of the second radiofrequency probe, the processor is configured to determine whether the second radiofrequency probe is one of the following probe configurations: a monopolar radiofrequency probe utilizing the third electrode; a bipolar radiofrequency probe coupled to the radiofrequency generator to form one channel and utilizing the third and fourth electrodes as a bipolar pair of electrodes; or a test probe.

In Example, 23, the tissue ablation system of Example 21, wherein the processor is further configured to: compare the measured impedance to one or more reference impedance values to determine the probe configuration of the second radiofrequency probe.

In Example 24, the tissue ablation system of Example 16, wherein the processor is further configured to: determine, based on the measured impedance, to which one of the plurality of electrical pins the one or more of the third and fourth electrodes are connected.

In Example 25, the tissue ablation system of Example 16, wherein the radiofrequency generator comprises switching elements configured to control radiofrequency power delivery.

In Example 26, the tissue ablation system of Example 25, wherein the processor is configured to execute program instructions to open and close the switching elements when measuring impedance between different electrical pins.

In Example 27, a multichannel radiofrequency tissue ablation system is provided. The system comprises: a radiofrequency generator comprising a power source and a controller operatively coupled to the power source and having a processor and a memory element. The radiofrequency generator has a plurality of connector ports, wherein each of the plurality of connector ports has a plurality of electrical pins. The system further comprises: a first radiofrequency probe electrically coupled to a first one of the plurality of connector ports to form a first channel and having first and second electrodes connected to corresponding ones of the plurality of electrical pins; a second radiofrequency probe electrically coupled to a second one of the plurality of connector ports to form a second channel and having third and fourth electrodes connected to corresponding ones of the plurality of electrical pins.

The power source can be configured to deliver radiofrequency power to the electrodes via the corresponding electrical pins. The processor is also configured to execute program instructions to: measure impedance between different electrical pins of the plurality of connector ports, and based on the measured impedance, determine configurations of the first and second radiofrequency probes.

In Example 28, the system of Example 27, wherein to determine the configurations of the radiofrequency probes, the processor is configured to determine whether the probes form one of the following configurations: two monopolar radiofrequency probes utilizing the first and third electrodes respectively; two bipolar radiofrequency probes utilizing the first and second electrodes as a first bipolar pair and the third and fourth electrodes as a second bipolar pair; or a test probe and a radiofrequency probe combination.

In Example 29, the system of Example 28, wherein the processor is further configured to compare the measured impedance to one or more reference impedance values to determine the probe configuration of the first and second radiofrequency probes.

In Example 30, the system of Example 28, wherein the processor is configured to determine, based on the measured impedance, to which ones of the plurality of electrical pins the first, second, third and fourth electrodes are connected.

In Example 31, the system of Example 27, wherein the radiofrequency generator comprises switching elements configured to control radiofrequency power delivery.

In Example 32, the system of Example 31, wherein the processor is configured to execute program instructions to open and close the switching elements when measuring impedance between different electrical pins.

In Example 33, a computer-implemented method of radiofrequency probe detection in a tissue ablation system is provided. The system comprises: a radiofrequency generator comprising a power source and a controller operatively coupled to the power source and having a processor and a memory element. The radiofrequency generator has a plurality of connector ports, wherein each of the plurality of connector ports has a plurality of electrical pins. The system further comprises a first radiofrequency probe electrically coupled to a first one of the plurality of connector ports of the radiofrequency generator to form a first channel and having first and second electrodes associated therewith, wherein each of the first and second electrodes are connected to the radiofrequency generator via a corresponding one of the plurality of electrical pins. The power source can be configured to deliver radiofrequency power to one of the first and second electrodes via the first one of the plurality of connector ports and the corresponding one of the plurality of electrical pins, the radiofrequency power being sufficient to provide controlled heating of tissue surrounding the at least one electrode during a surgical procedure. The method comprises executing via the processor, program instructions stored in the memory element to: measure an impedance between different electrical pins of one or more of the plurality of connector ports, and based on the measured impedance, determine a configuration of the first radiofrequency probe electrically coupled to the radiofrequency generator.

In Example 34, the computer-implemented method of Example 33, wherein to determine the configuration of the at least one radiofrequency probe electrically coupled to the radiofrequency generator, the method further comprises executing via the processor program instructions stored in the memory element to determine whether the first radiofrequency probe is: a monopolar radiofrequency probe utilizing the first electrode; a bipolar radiofrequency probe coupled to the radiofrequency generator to form one channel and utilizing the first and second electrodes configured as a bipolar pair of electrodes; or a test plug.

In Example 35, the computer-implemented method of Example 34, wherein the processor is further configured to: compare the measured impedance to one or more reference impedance values to determine the probe configuration of the first radiofrequency probe.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a schematic block diagram of an exemplary multichannel radiofrequency tissue ablation system in accordance with the teachings of the present invention.

FIG. 2 is a schematic block diagram of the controller of the multichannel radiofrequency tissue ablation system of FIG. 1 in accordance with the teachings of the present invention.

FIG. 3 is a schematic electrical diagram illustrating an exemplary use of controlling switching elements in an RF generator to detect automatically a radiofrequency probe configuration in accordance with the teachings of the present invention.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

A radiofrequency ablation (RFA) procedure is a minimally invasive medical procedure that is used to ablate targeted tissue at a surgical site, and typically involves using one or more energy delivery devices (e.g., radiofrequency (RF) probes) having one or more electrodes associated therewith. The radiofrequency probe can be coupled to one or more radiofrequency (RF) generators to deliver power in the form of radiofrequency energy to the targeted tissue via the electrodes. The radiofrequency energy generates localized heat which is used to target and destroy (e.g., ablate) the targeted tissue. In pain management, the targeted tissue can be nerve tissue responsible for sending pain signals.

The RFA procedure can be utilized to ablate different types of tissue, such as nerve tissue, adipose tissue, muscle tissue, and the like. The RFA procedure can be performed at various locations on an external surface of a subject or patient or within the subject. The RFA procedure performed within the subject can be performed in a minimally invasive manner (e.g., via a percutaneous, laparoscopic, endoscopic, or intravascular approach) or in a more invasive manner (e.g., via an open surgical procedure). For example, Applicant's existing technology (the Intracept® procedure by Relievant® Medsystems, Inc.) offers a safe and effective minimally invasive procedure that targets the basivertebral nerve (BVN) for the relief of chronic low back pain that originates at least partly from one or more endplates of one or more vertebral bodies. The Intracept® procedure involves application of radiofrequency energy from a radiofrequency generator using for example a bipolar radiofrequency probe, where the applied radiofrequency energy is sufficient to ablate the basivertebral nerve within the vertebral body. The procedure can be performed in multiple different vertebral bodies sequentially or simultaneously using a single radiofrequency probe or using multiple radiofrequency probes coupled to a single generator. The basivertebral nerve trunk can then be ablated by the RF probe. Other intraosseous nerves within the vertebral body that innervate the endplates and/or intervertebral disc can also be targeted and ablated.

The RFA procedure is typically performed using a surgical introducing or access assembly that can include a hollow cannula that forms a sheath and a stylet that is disposed within the cannula until a distal tip of the stylet extends beyond the cannula. The stylet adds rigidity and support to the cannula when inserted into the patient. The stylet helps the surgeon guide the assembly to the surgical site. Once the cannula is in position at the surgical site, then the stylet is removed from the cannula and the RF probe is inserted therein. The RF probe can then deliver RF energy to the targeted tissue at the surgical site. In a bipolar RF probe arrangement, the probe can include a pair of electrodes, including a first active energy delivering electrode and a second return electrode. According to another arrangement, the RF probes can be configured in a monopolar arrangement, where an RF probe employs an active energy delivering electrode and a separate device or element can be used as a return. The return can be configured as a return pad coupled to the subject or a return electrode disposed on a separate RF probe. For example, a first RF probe can be used that includes the active energy delivering electrode and a second RF probe can be used that includes a return electrode. The RF probe can also include additional structure, including, for example, temperature sensors (e.g., thermocouples or thermistors) that can be employed to sense or detect the temperature at the surgical site during the surgical procedure. The sensed or detected temperature can provide important information to the surgeon about the ablation procedure. The RFA procedure contemplates positioning the two electrodes of the one or more radiofrequency probes within the vertebral body. The RF generator supplies power to the active electrode of the RF probe at a selected power or voltage level and for a time sufficient to create a desired lesion within the vertebral body sufficient to ablate the basivertebral nerve within the vertebral body. In some implementations, the step of applying power to the RF probe includes supplying power for a selected duration or period of time (e.g., 1 minute to 1 hour or for any time within this range) to achieve a selected temperature at the surgical site. The temperature of the thermal energy supplied by the RF probe to the targeted tissue can range from between about 70 and about 115 degrees Celsius (e.g., from about 70 to about 90 degrees Celsius, from about 75 to about 90 degrees Celsius, from about 83 to about 87 degrees Celsius, from about 80 to about 100 degrees Celsius, from about 85 to about 95 degrees Celsius, from about 90 to about 110 degrees Celsius, from about 95 to about 115 degrees Celsius, or overlapping ranges thereof). The temperature ramp-up during the surgical procedure can range from between about 0.1-5 degrees Celsius/second (e.g., 0.1-1.0 degrees Celsius/second, 0.25 to 2.5 degrees Celsius/second, 0.5-2.0 degrees Celsius/second, 1.0-3.0 degrees Celsius/second, 1.5-4.0 degree Celsius/second, 2.0-5.0 degrees Celsius/second). The time of treatment can range from between about 10 seconds and about 1 hour (e.g., from 10 seconds to 1 minute, 1 minute to 5 minutes, from 5 minutes to 10 minutes, from 5 minutes to 20 minutes, from 8 minutes to 15 minutes, from 10 minutes to 20 minutes, from 15 minutes to 30 minutes, from 20 minutes to 40 minutes, from 30 minutes to 1 hour, from 45 minutes to 1 hour, or overlapping ranges thereof). Pulsed energy can be delivered as an alternative to or in sequence with continuous energy. For radiofrequency energy, the energy applied by the RF generator can range from between about 350 kHz and about 650 kHz (e.g., from 400 kHz to 600 kHz, from 350 kHz to 500 kHz, from 450 kHz to 550 kHz, from 500 kHz to 650 kHz, overlapping ranges thereof, or any value within the recited ranges, such as 450 kHz±5 kHz, 475 kHz±5 kHz, 487 kHz±5 kHz). The power of the radiofrequency energy generated by the RF generator can range from between about 5 W and about 30 W (e.g., from 5 W to 15 W, from 5 W to 20 W, from 8 W to 12 W, from 10 W to 25 W, from 15 W to 25 W, from 20 W to 30 W, from 8 W to 24 W, and overlapping ranges thereof, or any value within the recited ranges).

Although described primarily in connection with procedures for the treatment within the spine, the systems and methods of the present invention can also be used for radiofrequency tissue ablation procedures intended to treat ailments or conditions other than those associated with the spine or back pain. The procedures can involve ablation of nerves outside of bones but related to the spine (e.g., sacroiliac joints, facet joints, etc.). Target treatment locations within bones other than vertebral bodies may also be accessed. For example, target treatment locations within a humerus, radius, femur, tibia, calcaneus, tarsal bones, hips, knees, phalanges, and/or other orthopedic targets can be accessed. The ablation procedures can include, for example, ablation of nerves within or surrounding other bones other than the vertebral column, cardiac tissue ablation for treatment of atrial fibrillation or other abnormal heart rhythms irregularities, tumor ablation at any location within the body (e.g., within bones, lungs, breasts, thyroids, livers, or other organs or tissues), peripheral nerve ablation, pulmonary artery ablation, renal denervation procedures, uterine fibroid ablation, endometrial ablation, and/or the like. The systems and methods described herein may be used in connection with any radiofrequency procedure during which impedance (e.g., tissue impedance or impedance between two electrodes) is monitored.

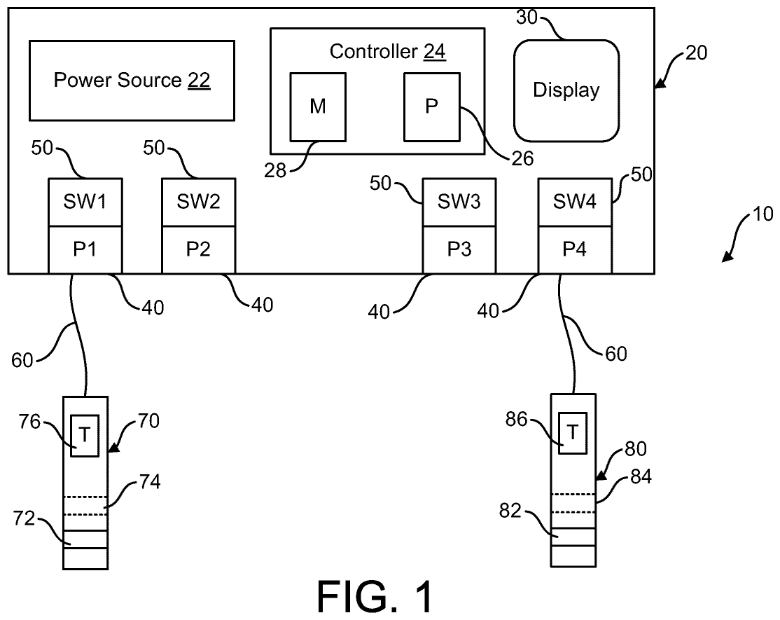

FIG. 1 is a schematic block diagram of a tissue ablation system 10 suitable for use with the present invention. The tissue ablation system 10 includes a generator, such as a radiofrequency generator 20, that can be coupled to one or more radiofrequency probes. The RF generator 20 can be electrically and communicatively coupled to the RF probes 70, 80 via any suitable conduit 60. The conduits 60 can facilitate bidirectional communication of electrical signals and data or instructions between the RF generator 20 and the RF probes 70, 80. For example, the conduits 60 can include one or more electrical wires or lines (not shown) suitable for conveying the electrical signals. The generator can also be any other type of energy source suitable for supplying ablative energy to the probes.

According to one embodiment, the illustrated RF generator 20 can include a power source 22 that can be configured to generate and supply radiofrequency power or energy to the RF probes 70, 80 via the conduits 60 at a desired frequency and power level. The RF generator 20 can also include a controller 24 that includes for example a processor (e.g., CPU) 26 suitable for processing and executing applications and instructions that are stored in a memory element 28. The processor 26 can be any suitable type of processor, such as a special purpose processor, and the memory element 28 can be any suitable type of memory. The processor 26 can be implemented in hardware, software, firmware, or any suitable combination thereof, and can be configured to execute program instructions stored in the memory element 28. The processor 26 and the memory element 28 are shown herein as forming part of the controller 24, but one of ordinary skill in the art will readily recognize that the processor 26 and the memory 28 can form part of other components of the RF generator 20. For the sake of simplicity, only one processor 26 and memory element 28 are shown, although multiple processors and memory elements can also be employed in the RF generator 20. The controller 24 can be configured as described herein, and at least is configured to monitor temperature, power, and/or impedance in connection with a tissue ablation procedure performed by the tissue ablation system 10. The programming instructions can be implemented in C, C++, JAVA, or any other suitable programming languages. In some embodiments, some or all of the portions of the controller 24 can be implemented in application specific circuitry such as ASICs and FPGAs.

The RF generator 20 can further include a user interface or display element 30 suitable for displaying information or instructions to a user (e.g., surgeon) via a series of user interfaces or for allowing the user to provide instructions to the RF generator. As such, the display element 30 can employ a user interface generator (not shown) for generating the user interfaces based on programmatic instructions stored in the memory element 28 and processed by the processor 28 or by a processor forming part of the display element 30. The display element 30 can be configured to display information to the user. For example, during startup and use, the current status of the RF generator 20 and energy delivery or treatment parameters can be displayed on the display element 30. During energy delivery, the display element 30 can be configured to display desired treatment time, remaining treatment time, temperature, impedance, and power information (alphanumerically and/or graphically). For example, graphical representations of power vs. time and impedance vs. time can be displayed. The display element 30 can also be separate from the RF generator 20 and need not form part of the generator. Although not shown, the tissue ablation system 10 can also employ additional accessory devices, such as suitable user input devices including a keyboard, mouse, trackpad, voice-activated input device, and the like.

The illustrated RF generator 20 can also have formed therein a plurality or series of connector ports 40 that allow the RF probes 70, 80 to be coupled to the generator. The RF generator 20 can have any selected number of connector ports 40. In the illustrated embodiment, the RF generator 20 includes four connector ports 40. The connector ports 40 can have any selected size, shape, and configuration. According to one embodiment, the connector ports 40 can employ a series of electrical contacts or pins that connect to RF probes and allow electrical energy and data to be exchanged therebetween. For example, a first electrical pin (or multiple electrical pins) can be coupled to the RF probe so as to deliver RF power to an energy delivering electrode when placed thereon and a second electrical pin can communicate with a return electrode when placed thereon. A third electrical pin can be employed to communicate with a temperature sensor in the RF probe and a fourth electrical pin can be used to exchange data between the RF generator and the RF probe. Those of ordinary skill in the art will readily recognize that any suitable electrical connection type (e.g., Lemo or Din type connectors), arrangement, and number of electrical connectors can be employed in the connector ports 40.

The illustrated RF generator 20 can also employ a switching element module or a series of discrete switching elements 50 that can be used to help control the generation, modulation, and delivery of RF energy. The switching elements 50 help ensure the precise delivery of energy to the target tissue via the RF probes while maintaining system safety and functionality. The switching elements 50 can also control the RF energy delivered to the active energy delivering electrode of the RF probe through an associated connector port 40. For the sake of simplicity, a switching element 50 is shown coupled to and disposed in electrical communication with each illustrated connector port 40. Those of ordinary skill in the art will readily recognize that a separate switching element module that is arranged in electrical communication with each of the connector ports 40 can also be employed. The illustrated switching elements 50 can also be positioned and arranged to manage the activation of circuits connected to sensors in the RF probes 70, 80, such as temperature sensors or impedance measurement systems. The switching elements can also be arranged to route sensor signals to specific monitoring circuits in the controller 24 for processing. The switching elements 50 can also serve to verify proper grounding or return electrode connection before enabling RF output to ensure patient safety. The switching elements 50 can be any suitable type of switching element, such as MOSFETs, IGBTs, Pin diodes, mechanical and solid-state relays (SSR), and the like. For example, two sets of relays can be provided on both the power and ground side for each connection point.

The RF probe configurations which can be connected to the illustrated tissue ablation system 10 depend upon the selected type of surgical procedure being performed and/or the purpose and function of the RF probe. The RF probe 70 has a main body having an outer surface having one or more electrodes mounted thereon. In a bipolar RF probe arrangement, the RF probe 70 can have a first active energy delivering electrode 72 and a return electrode 74 disposed on an outer surface of the main body. The probe can also include a temperature sensor 76 for sensing or measuring temperature at the surgical site. The electrodes and the temperature sensor 76 can be coupled to the RF generator 20 via electrical leads or wires that pass through the conduit 60. If the RF probe 70 is constructed as a monopolar device, then the probe can include a single active electrode 72, which can function as the active energy delivering electrode or as the return electrode. In this type of arrangement, the second electrode 74 (in dashed lines) is either inactivated or not present. Similarly, the RF probe 80 has a main body having an outer surface having one or more electrodes mounted thereon. In a bipolar RF probe arrangement, the RF probe 80 can also have a first active energy delivering electrode 82 and a return electrode 84. The probe can also include a temperature sensor 86. The electrodes and the sensor can also be coupled to the RF generator 20 via electrical leads that pass through the conduit 60. If the RF probe 80 is constructed as a monopolar device, then the probe can also include a single active electrode 82, which can function as the active energy delivering electrode or as the return electrode. In this type of arrangement, the second electrode 84 is either inactivated or not present. Each RF probe that is coupled to a connector port 40 of the RF generator 20 forms in essence a channel. Multiple RF probes coupled to multiple connector ports 40 of the RF generator 20 can form multiple channels. Bipolar RF probes can be designed to require one or two ports for one device. As such, one bipolar RF probe can form one or two channels. So, there is a lot of different probe configurations possible. The multichannel radiofrequency tissue ablation system 10 can be provided with a test plug.

The systems and methods described herein are generally aimed at providing probe detection in multichannel radiofrequency tissue ablation systems to automatically determine unique RF probe configurations and wiring schematics without user intervention. The probe detection approach centers on systematic impedance measurements between electrical pins to create characteristic patterns that identify probe types and connections.

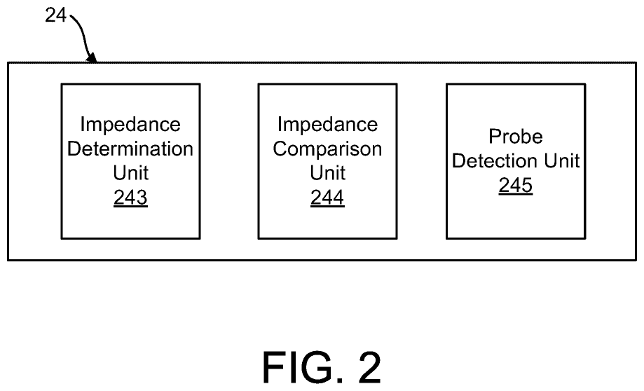

For implementing methods of probe detection in the multichannel radiofrequency tissue ablation system 10, the controller 24 can include an impedance determination unit 243, an impedance comparison unit 244, and a probe detection unit 245, as shown for example in FIG. 2. The impedance comparison unit 244 can form part of the illustrated impedance determination unit 243 and/or the probe detection unit 245 rather than be a separate component or unit. For the sake of simplicity and the ease of discussion, the impedance comparison unit 244 is shown as a separate unit.

The impedance determination unit 243 of the controller 24 can be configured to measure and determine impedance between specific electrical contacts or pins of the connector ports 40 on one or multiple channels so as to determine whether there is any measurable impedance and if so, the magnitude or value of the measured impedance. The impedance determination unit 243 can be configured to open and close the switching elements 50 for measuring the impedance between the individual electrical pins of the connector ports 40. The impedance determination unit 243 can be configured to open and close different ones of the switching elements 50 that can be electrically coupled to specific electrical contacts or pins of one or more of the connector ports 40, and then measure the impedance associated therewith. The impedance determination unit 243 can be configured to evaluate if the electrical connection via the switching elements are open (e.g., no impedance measured) or closed (e.g., measurable impedance).

The impedance comparison unit 244 of the controller 24 can be configured to compare the measured impedance to prestored impedance values or ranges of values of a reference RF probe configuration for determining an electrode arrangement or configuration of any RF probe that is electrically coupled to the RF generator 20. The reference impedance values or ranges of impedance values can be preset and stored in the memory element 28 of the controller 24 in any selected format, such as in a tabular format. For example, the reference impedance values or ranges of values can be preset and stored for the following reference RF probe configurations: a monopolar RF probe, a bipolar RF probe (single or dual channel type), and a test plug or probe. For example, the test plug can be a relieving bipolar RF probe and/or have a set or predetermined impedance value (e.g., 100 ohms).

The probe detection unit 245 of the controller 24 can be configured to determine, based on the measured impedance from the impedance determination unit 243 and the comparison by the impedance comparison unit 244, a specific configuration of any RF probe(s) electrically coupled to the RF generator 20. In one example, the probe detection unit 245 can be configured to determine whether the connected RF probe is being used as the monopolar RF probe, the bipolar RF probe (single or dual channel type), or the test plug. The probe detection unit 245 can be further configured to determine, based on the measured impedance, the specific electrical pins that the one or more electrodes of the RF probe are connected, in order to determine wiring schematics for the particular RF probe configuration arrangement in the system.

The RF generator can be adapted to configure itself to a specific therapy delivery based on the detection of a type of probe configuration connected to the RF generator 20 and, optionally, based on the determined wiring schematics.

The probe detection unit 245 can use prestored or predetermined truth or logic tables to define the expected impedance patterns for different probe configurations. The truth tables can establish relationships between three tests: measuring impedance between pins within Channel 1 (Test A), measuring impedance between pins within Channel 2 (Test B), and measuring impedance between Channel 1 and Channel 2 (Test C). Each probe type—monopolar, bipolar, or test plug—creates a combination of “OPEN” and “CLOSED” electrical states across the different tests. The “OPEN” state means or refers to no impedance measured or detected, and the “CLOSED” state means or refers to a positive impedance reading or measurement. It should be understood, however, that if more than two channels are provided in the multichannel RF tissue ablation system, the number of tests can increase accordingly to perform measurements for all the channels and between all the channels available. The truth tables can be stored in the memory 28.

For implementation in the multichannel RF tissue ablation system, sets of switching elements 50, such as solid-state relays (SSR), can form part of a switching element module that can control impedance measurements between different combinations of pins from a single connector port 40 or between multiple connector ports 40. The RF generator 20 sequences through measurements according to the predefined truth tables, comparing impedance or electrical measurement or detection results against prestored values to identify the type of probe configuration. The RF generator 20 thus enables automated detection and determination of both single-probe and dual-probe electrode or electrical arrangements without requiring manual verification or detection steps.

Below are examples of specific embodiments or illustrative use cases for carrying out the present invention. The examples are offered for illustrative purposes only and are not intended to limit the scope of the present invention.

The following examples demonstrate specific implementations of the foregoing measurement and detection principles. Example 1 details single-probe detection using a three-test sequence. Example 2 expands this approach to dual-probe configurations. Example 3 illustrates the SSR control system that enables precise impedance measurements.

### Example 1

In one example of implementation of the automatic determination of probe arrangement principles according to the present invention, the multichannel tissue ablation system can have an RF generator 20 comprising multiple connector ports 40 to form at least Channel 1 and Channel 2. Each connector port 40 can include any selected number of electrical pins. According to one embodiment, the connector port includes four electrical pins, including Pin 1, Pin 2, Pin 3 and Pin 4. In the current example, a single RF probe having a bipolar RF probe arrangement or configuration and being used as such is connected to (e.g., plugged into) one of the connector ports 40 of the RF generator 20, with Pin 1 of Channel 1 electrically coupled to the power source 22 and to one or more of the electrodes 72, 74 of the bipolar RF probe 70, and Pin 4 of Channel 1 electrically connected to ground and to one or more of the electrodes 72, 74 of the bipolar RF probe.

For automatically determining the arrangement or configuration of the attached RF probes, the controller 24 can automatically perform an impedance measurement via the impedance determination unit 243 by performing a three-test sequence by switching one or more of the switching elements 50 on and off. The impedance determination unit 243 can measure the impedance of the probe by measuring impedance between selected pins of the connector ports 40. The impedance comparison unit 244 that compares the measured impedance with one or more values set forth in Table 1. Table 1 shows a truth or logic table for the three example impedance measurement tests (e.g., Test A, Test B, and Test C). Test A relates to measurement of Pin 1 to Pin 4 impedance of Channel 1. Test B relates to measurement of Pin 1 to Pin 4 impedance of Channel 2. Test C relates to measurement of Pin 1 of Channel 1 to Pin 4 of Channel 2 impedance. The truth table (e.g., Table 1) indicates “CLOSED” (e.g., in case there is an impedance measured) vs “OPEN” (e.g., no impedance measured) and illustrates in table format the test results to be used by the controller 24 when automatically determining the electrode configuration of the RF probe coupled to the RF generator 20.

TABLE 1

| Test | Test A | Test B | Test C |
|---|---|---|---|
| Monopolar RF probe | OPEN | OPEN | OPEN |
| Bipolar RF probe | CLOSED | OPEN | OPEN |
| Test Plug (relieving | 100 ohms | OPEN | OPEN |
| bipolar probe) | | | |

The example truth table (Table 1) sets forth distinct impedance measurement patterns and switching element positions for each type of electrode arrangement or configuration, during use, of the RF probe 70. By simple way of example, and as shown in Table 1, for a monopolar RF probe arrangement, all three tests show no electrical connection (“OPEN”) between selected pins as there is no completed electrical circuit between any of the tested pin combinations. More specifically, an associated switching element is disposed in an OPEN position. When a bipolar RF probe is connected, Test A indicates an electrical connection (“CLOSED”) between Pin 1 and Pin 4 of Channel 1 due to the bipolar electrode pair arrangement, while Tests B and C show no connection (“OPEN”) as expected with no device on Channel 2 and no cross-channel connections. The OPEN notation indicates that a switching element is open, and the CLOSED notation indicates that the switch element is closed.

In the case of a test plug, Test A measures precisely 100 ohms between Pin 1 and Pin 4 of Channel 1, representing the characteristic impedance of the test device. Tests B and C show no connection (“OPEN”), confirming the test plug is only connected to Channel 1.

Based on the test results shown in Table 1, the controller is able to automatically detect that a specific RF probe of bipolar type is plugged into the RF generator and differentiate it from the test plug, as well as determine a specific wiring schematic of the bipolar RF probe connection by indicating the two electrodes of the bipolar RF probe connected to Pin 1 and Pin 4 of Channel 1.

As illustrated by Example 1, through impedance measurement patterns defined using truth tables, the controller can identify which type of probe is connected to the generator without requiring any manual configuration steps or user input. The combination of measured impedance values creates a signature for each probe type, enabling automated detection and appropriate system configuration.

### Example 2

This example illustrates an embodiment of the multichannel RF tissue ablation system with two RF probes plugged into the RF generator 20 comprising two ports (Channel 1 and Channel 2) each comprising four electrical pins (Pin 1, Pin 2, Pin 3, and Pin 4).

Table 2 shows a truth or logic table showing possible results of probe detection for three example impedance measurement tests (e.g., Test A, Test B, Test C) when two RF probes are plugged into the RF generator 20. As in Example 1, Test A relates to measurement of Pin 1 to Pin 4 impedance, Channel 1. Test B relates to measurement of Pin 1 to Pin 4 impedance, Channel 2. Test C relates to measurement of Pin 1, Channel 1 to Pin 4, Channel 2 impedance. The truth table indicates “CLOSED” state in case there is reading of impedance vs “OPEN” state if no impedance is detected.

As shown in the example truth table (Table 2), for a monopolar RF probe arrangement, Tests A and B show no connection (“OPEN”) between Pin 1 and Pin 4 within their respective channels, while Test C indicates a connection (“CLOSED”) between Pin 1 of Channel 1 and Pin 4 of Channel 2, demonstrating the characteristic cross-channel circuit completion required for monopolar operation.

For two bipolar RF probes arrangement, Test A and B both show connections (“CLOSED”) between Pin 1 and Pin 4 within their respective channels due to the paired electrodes on each probe, while Test C confirms isolation between channels by showing no connection (“OPEN”) between Channel 1 and Channel 2.

For a test plug configuration alongside another probe, Test A measures 100 ohms between Pin 1 and Pin 4 of Channel 1, Test B shows a connection (“CLOSED”) on Channel 2, and Test C verifies channel isolation by showing no connection (“OPEN”) between the Channels 1 and 2. This unique pattern definitively identifies the presence of both the test plug and second probe in the system.

TABLE 2

| Test | Test A | Test B | Test C |
|---|---|---|---|
| Monopolar RF probe | OPEN | OPEN | CLOSED |
| Bipolar RF probe | CLOSED | CLOSED | OPEN |
| Test Plug (relieving | 100 ohms | CLOSED | OPEN |
| bipolar probe) | | | |

Based on the example test results shown in Table 2, the controller 24 is able to automatically detect more than one specific RF probe in case two or more RF probes are plugged into the RF generator and differentiate them from the test plug, as well as determine specific wiring schematics of each RF probe connection.

### Example 3

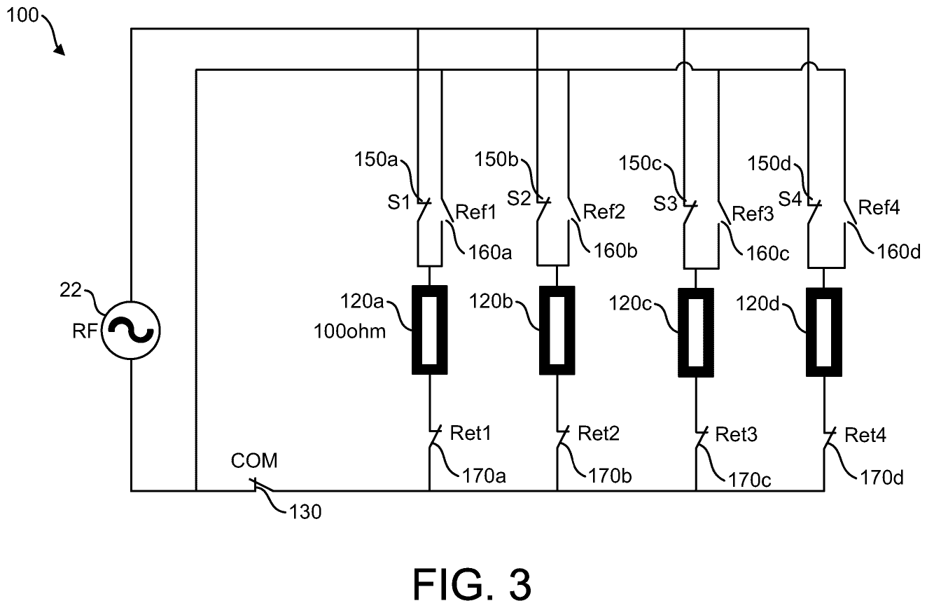

FIG. 3 shows a schematic electrical diagram or circuit 100 illustrating the exemplary simplified connections of switching elements (e.g., SSR switching elements) associated with the electrodes of the RF probes 70, 80 in the multichannel RF tissue ablation system 10, in accordance with the teachings of the present invention. The illustrated circuit 100 includes an RF power source 22 and a common connection point (COM) 130. The circuit 100 can include four source switching elements or relays 150a-150d (S1-S4) paired with corresponding return switching elements or relays 170a-170d (Ret1-Ret4) disposed in a parallel arrangement. Each circuit or measurement path formed by the switching elements can also include a 100-ohm impedance element 120a-120d and additional reference switches 160a-160d (Ref1-Ref4). The circuit diagram 100 illustrates an example electrical circuit architecture where pairs of SSRs relays (S1/Ret1, S2/Ret2, S3/Ret3, S4/Ret4) control the electrical pathways between the RF source 22 and the probe electrodes.

The RF power source 22 connects to a series of parallel circuits, each controlled by a pair of switching elements (S1/Ret1, S2/Ret2, S3/Ret3, S4/Ret4). The common connection point (COM) 130 serves as the reference point for all measurements and helps establish or allow current to flow along the electrical return pathways that include the return switching elements 160a-160d. Further, when the return switching elements 160a-160d are open, then current does not pass along those specific circuit pathways. Each illustrated circuit pathway can optionally include the 100-ohm resistor or impedance element 120a-120d, representing the characteristic impedance used for system calibration and verification.

The pairs of switching elements (S1/Ret1, S2/Ret2, S3/Ret3, S4/Ret4) in each parallel circuit pathway can be arranged in a symmetrical configuration, with each pair consisting of a source relay (S1-S4) and a return relay (Ret1-Ret4). This paired switching arrangement enables complete circuit isolation during measurements. Reference switches (Ref1-Ref4) provide additional control points for the measurement system, enabling precise impedance measurements between any combination of electrodes.

The physical layout of the circuit 100 demonstrates the system's ability to independently control and measure each electrode pathway. The parallel arrangement of the SSR pairs (S1/Ret1, S2/Ret2, S3/Ret3, S4/Ret4) enables sequential testing of different pin combinations while maintaining electrical isolation between unused paths. This isolation is for accurate impedance measurements and proper probe detection.

Each SSR pair forms a complete measurement circuit when closed. The source relays control the connection to the RF source 22, while the return relays complete the circuit pathways. This dual-relay approach provides redundant isolation, preventing any unintended current paths that could interfere with measurements or compromise safety.

Table 3 provides a matrix of switching element states during operation, illustrating that one pair of SSRs can be closed at any given time. For example, when the controller 24 activates or closes the S1/Ret1 150a, 170a pair, all other pairs of switching elements (S2/Ret2, S3/Ret3, and S4/Ret4) remain open. This enables the impedance determination unit to measure and determine the impedance along the associated electrical or circuit pathway, which reflects the circuit path through the first SSR pair. The controller 24 maintains this activation pattern throughout the measurement sequence.

Similarly, when the controller 24 activates the S2/Ret2 pair of switching elements 150, 170b, the remaining pairs of relays S1/Ret1, S3/Ret3, and S4/Ret4 remain open. This sequential isolation continues for the S3/Ret3 and S4/Ret4 switching elements, creating an impedance measurement technique that measure the impedance along each potential circuit pathway.

TABLE 3

| SSR | S1/Ret1 | S2/Ret2 | S3/Ret3 | S4/Ret4 |
|---|---|---|---|---|
| S1/Ret1 | CLOSED | OPEN | OPEN | OPEN |
| S2/Ret2 | OPEN | CLOSED | OPEN | OPEN |
| S3/Ret3 | OPEN | OPEN | CLOSED | OPEN |
| S4/Ret4 | OPEN | OPEN | OPEN | CLOSED |

Table 3 demonstrates how this activation principle extends across all four pairs of switching elements. Each row in Table 3 represents a complete state of the circuit 100 during one impedance measurement phase, with the “CLOSED” state indicating the active pair of switches and “OPEN” state indicating all other inactive pairs of switches. This visualization helps enable proper implementation of the control logic.

The measurement patterns shown in the table enables systematic evaluation of all possible connections while maintaining measurement integrity. This approach enables the system to build a complete profile of the connected probes through sequential impedance measurements, forming the probe detection capabilities described in Examples 1 and 2.

The controlled switching sequence, combined with precise impedance measurements, allows the system to accurately determine probe configurations without requiring manual intervention. The SSR control system translates the theoretical detection principles into practical, reliable probe identification capabilities.

Beyond the basic switching functions, the arrangement of switching elements also provides inherent safety features. The dual-relay design enables fail-safe operation, as both relays in a pair must be properly closed to complete a circuit. This redundancy helps prevent unintended RF energy delivery and enables patient safety throughout the procedure. The system's ability to control individual SSR pairs with precise timing enables sophisticated measurement sequences. The controller 24 can adapt the measurement pattern based on initial results, implementing more detailed testing when needed to definitively identify complex probe configurations.

In some embodiments, the system 10 can perform impedance measurements in a defined sequence. For example, each SSR pair maintains closure for a selected period time, such as for example 2 ms, during measurement. Further, a 0.5 ms delay occurs between switching pairs to allow for signal stabilization. The controller 24 executes three measurement attempts before declaring a reading valid. If unclear readings occur, the sequence repeats up to two additional times.

In some embodiments, the system can monitor for deviations from expected impedance patterns. When SSR failures occur, the controller disables the affected pair and reroutes measurements through alternate paths. The system logs measurement interruptions and resumes from the last valid state. Error codes display on a user interface to indicate specific fault conditions.

In some embodiments, the probe detection method can extend to systems with four or more channels. Additional truth tables incorporate new pin combinations. The controller optimizes measurement sequences based on active channel count. Parallel processing enables simultaneous measurements across channel groups.

In some embodiments, the controller can perform continuous impedance monitoring during therapy delivery. Redundant SSR pairs provide backup switching paths. The system disables RF output upon detecting probe disconnection. Pre-therapy verification confirms proper probe identification before enabling power delivery.

In some embodiments, the system can adjust measurement timing based on therapy phase. During initial probe detection, measurements occur more frequently. Once therapy begins, the system reduces measurement frequency to minimize interference with power delivery while maintaining sufficient monitoring for safety verification.

Any methods described herein may be embodied in, and partially or fully automated via, software code modules (e.g., in the form of an algorithm or machine-readable instructions) stored in a memory element such as a tangible, non-transitory computer-readable medium executed by one or more processors or other computing devices. The software may be downloaded to a processor in electronic form. In embodiments involving multiple processors, the processors may operate in parallel to form a parallel processing system in which a process is split into parts that execute simultaneously on different processors of the ablation system. The methods may be executed on the computing devices in response to execution of software instructions or other executable machine-readable code read from the memory or tangible computer readable medium. A tangible computer readable medium is a data storage device that can store data that is readable by a computer system. Examples of computer readable mediums include read-only memory (e.g., ROM or PROM, EEPROM), random-access memory, other volatile or non-volatile memory devices, CD-ROMs, magnetic tape, flash drives, and optical data storage devices.

As will be appreciated by a person of ordinary skill in the art, computer-executable instructions stored in tangible computer storage media define specific functions to be performed by computer hardware such as computer processors. In general, in such an implementation, the computer-executable instructions are loaded into memory accessible by at least one computer processor (for example, a programmable microprocessor or microcontroller or an application specific integrated circuit). The at least one computer processor then executes the instructions, causing computer hardware to perform the specific functions defined by the computer-executable instructions. As will be appreciated by a person of ordinary skill in the art, computer execution of computer-executable instructions is equivalent to the performance of the same functions by electronic hardware that includes hardware circuits that are hardwired to perform the specific functions. As such, while embodiments illustrated herein are typically implemented as some combination of computer hardware and computer-executable instructions, the embodiments illustrated herein could also be implemented as one or more electronic circuits hardwired to perform the specific functions illustrated herein.

Although certain embodiments and examples have been described herein, aspects of the methods and devices shown and described in the present disclosure may be differently combined and/or modified to form still further embodiments. Optional features of various device and system embodiments may be included in some embodiments and not in others. Additionally, the methods described herein may be practiced using any device suitable for performing the recited steps. Further, the disclosure (including the figures) herein of any particular feature, aspect, method, property, characteristic, quality, attribute, element, or the like in connection with various embodiments can be used in all other embodiments set forth herein. Any section headings used herein are merely provided to enhance readability and are not intended to limit the scope of the embodiments disclosed in a particular section to the features or elements disclosed in that section.

While the embodiments are susceptible to various modifications, and alternative forms, specific examples thereof have been shown in the drawings and are herein described in detail. It should be understood, however, that the embodiments are not to be limited to the particular forms or methods disclosed, but to the contrary, the embodiments are to cover all modifications, equivalents, and alternatives falling within the spirit and scope of the various embodiments described and the appended claims. Any methods disclosed herein need not be performed in the order recited and in other alternative embodiments one or more method steps may be skipped altogether.

Various embodiments of the disclosure have been presented in a range format. It should be understood that the description in range format is merely for convenience and brevity and should not be construed as an inflexible limitation on the scope of the invention. The ranges disclosed herein encompass any and all overlap, sub-ranges, and combinations thereof, as well as individual numerical values within that range. For example, description of a range such as from 70 to 115 degrees should be considered to have specifically disclosed subranges as well as individual numbers within that range, for example, 70, 70.5, 80, 115, and any whole and partial increments therebetween. Language such as “at least,” “greater than,” “less than,” “between,” and the like includes the number recited. Numbers preceded by a term such as “about” (in the meaning of “approximately”) include the recited numbers. For example, “between about 5 W and about 30 W” includes 5 W and 30 W. As used herein in the specification and claims, including as used in the examples and unless otherwise expressly specified, all numbers may be read as if prefaced by the word “about” even if the term does not expressly appear. The phrase “about” may be used when describing magnitude to indicate that the value described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “70” is disclosed, then “about 70” is also disclosed. Any numerical range recited herein is intended to include all sub-ranges subsumed therein.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

What is claimed is:

1. A tissue ablation system, comprising:
a radiofrequency generator having a power source and a controller operatively coupled to the power source and having a processor and a memory element, the radiofrequency generator having a plurality of connector ports, wherein each of the plurality of connector ports has a plurality of electrical pins;
a first radiofrequency probe electrically coupled to a first one of the plurality of connector ports of the radiofrequency generator to form a first channel and having first and second electrodes associated therewith, wherein each of the first and second electrodes are connected to the radiofrequency generator via a corresponding one of the plurality of electrical pins,
wherein the power source is configured to deliver radiofrequency power to one of the first and second electrodes via the first one of the plurality of connector ports and the corresponding one of the plurality of electrical pins, the radiofrequency power being sufficient to provide controlled heating of tissue surrounding the at least one electrode during a surgical procedure;
wherein the processor is configured to execute program instructions stored in the memory element, such that the program instructions, when executed, cause the processor to:
measure an impedance between different electrical pins of one or more of the plurality of connector ports, and
based on the measured impedance, determine a configuration of the first radiofrequency probe electrically coupled to the radiofrequency generator.

2. The system of claim 1, wherein, to determine the configuration of the first radiofrequency probe, the processor is configured to determine whether the first radiofrequency probe is one of the following probe configurations:
a monopolar radiofrequency probe utilizing the first electrode and a common return electrode;
a bipolar radiofrequency probe coupled to the radiofrequency generator to form one channel and utilizing the first and second electrodes as a bipolar pair of electrodes;
a bipolar radiofrequency probe utilizing two monopolar radiofrequency electrodes, wherein one of the two monopolar radiofrequency electrodes is connected to the first electrode and the other of the two monopolar electrodes is connected to the second electrode to form a bipolar pair where current flows between the first and second electrodes; or
a test probe.

3. The system of claim 2, wherein the test probe has a predetermined impedance value.

4. The system of claim 2, wherein the processor is further configured to compare the measured impedance to one or more reference impedance values to determine the probe configuration of the first radiofrequency probe.

5. The system of claim 1, wherein the processor is further configured to determine, based on the measured impedance, to which one of the plurality of electrical pins the one or more of the first and second electrodes are connected.

6. The system of claim 1, further comprising:
a second radiofrequency probe electrically coupled to a second one of the plurality of connector ports of the radiofrequency generator to form a second channel and having third and fourth electrodes associated therewith, wherein each of the third and fourth electrodes are connected to the radiofrequency generator via a corresponding one of the plurality of electrical pins.

7. The system of claim 6, wherein to determine the configuration of the second radiofrequency probe, the processor is configured to determine whether the second radiofrequency probe is one of the following probe configurations:
a monopolar radiofrequency probe utilizing the third electrode;
a bipolar radiofrequency probe coupled to the radiofrequency generator to form one channel and utilizing the third and fourth electrodes as a bipolar pair of electrodes; or
a test probe.

8. The system of claim 6, wherein the processor is further configured to:
compare the measured impedance to one or more reference impedance values to determine the probe configuration of the second radiofrequency probe.

9. The system of claim 1, wherein the processor is further configured to:
determine, based on the measured impedance, to which one of the plurality of electrical pins the one or more of the third and fourth electrodes are connected.

10. The system of claim 1, wherein the radiofrequency generator comprises switching elements configured to control radiofrequency power delivery.

11. The system of claim 10, wherein the processor is configured to execute program instructions to open and close the switching elements when measuring impedance between different electrical pins.

12. A multichannel radiofrequency tissue ablation system, comprising:
a radiofrequency generator comprising a power source and a controller operatively coupled to the power source and having a processor and a memory element, the radiofrequency generator having a plurality of connector ports, wherein each of the plurality of connector ports has a plurality of electrical pins;
a first radiofrequency probe electrically coupled to a first one of the plurality of connector ports to form a first channel and having first and second electrodes connected to corresponding ones of the plurality of electrical pins;
a second radiofrequency probe electrically coupled to a second one of the plurality of connector ports to form a second channel and having third and fourth electrodes connected to corresponding ones of the plurality of electrical pins;
wherein the power source is configured to deliver radiofrequency power to the electrodes via the corresponding electrical pins;
wherein the processor is configured to execute program instructions to:
measure impedance between different electrical pins of the plurality of connector ports, and
based on the measured impedance, determine configurations of the first and second radiofrequency probes.

13. The system of claim 12, wherein to determine the configurations of the radiofrequency probes, the processor is configured to determine whether the probes form one of the following configurations:
two monopolar radiofrequency probes utilizing the first and third electrodes respectively;
two bipolar radiofrequency probes utilizing the first and second electrodes as a first bipolar pair and the third and fourth electrodes as a second bipolar pair; or
a test probe and a radiofrequency probe combination.

14. The system of claim 13, wherein the processor is further configured to compare the measured impedance to one or more reference impedance values to determine the probe configuration of the first and second radiofrequency probes.

15. The system of claim 13, wherein the processor is configured to determine, based on the measured impedance, to which ones of the plurality of electrical pins the first, second, third and fourth electrodes are connected.

16. The system of claim 12, wherein the radiofrequency generator comprises switching elements configured to control radiofrequency power delivery.

17. The system of claim 16, wherein the processor is configured to execute program instructions to open and close the switching elements when measuring impedance between different electrical pins.

18. A computer-implemented method of radiofrequency probe detection in a tissue ablation system, the system comprising: a radiofrequency generator comprising a power source and a controller operatively coupled to the power source and having a processor and a memory element, the radiofrequency generator having a plurality of connector ports, wherein each of the plurality of connector ports has a plurality of electrical pins; and a first radiofrequency probe electrically coupled to a first one of the plurality of connector ports of the radiofrequency generator to form a first channel and having first and second electrodes associated therewith, wherein each of the first and second electrodes are connected to the radiofrequency generator via a corresponding one of the plurality of electrical pins, wherein the power source is configured to deliver radiofrequency power to one of the first and second electrodes via the first one of the plurality of connector ports and the corresponding one of the plurality of electrical pins, the radiofrequency power being sufficient to provide controlled heating of tissue surrounding the at least one electrode during a surgical procedure, the method comprising executing via the processor program instructions stored in the memory element to:
measure an impedance between different electrical pins of one or more of the plurality of connector ports, and
based on the measured impedance, determine a configuration of the first radiofrequency probe electrically coupled to the radiofrequency generator.

19. The computer-implemented method of claim 18, wherein to determine the configuration of the at least one radiofrequency probe electrically coupled to the radiofrequency generator, the method further comprises executing via the processor program instructions stored in the memory element to determine whether the first radiofrequency probe is:
a monopolar radiofrequency probe utilizing the first electrode;
a bipolar radiofrequency probe coupled to the radiofrequency generator to form one channel and utilizing the first and second electrodes configured as a bipolar pair of electrodes; or
a test plug.

20. The computer-implemented method of claim 19, wherein the processor is further configured to:
compare the measured impedance to one or more reference impedance values to determine the probe configuration of the first radiofrequency probe.
